(19) United States
(12) Patent Application Publication
(10) Pub. No.: US 2007/0276178 A1
(43) Pub. Date: Nov. 29, 2007

(54) ANATOMIC PROSTHESIS FOR TREATING HERNIAS, SUCH AS INGUINAL AND CRURAL AND OTHER HERNIAS

(75) Inventors: Patrick CARTERON, Chalain le Comtal (FR); William WIECEK, Bonson (FR)

Correspondence Address: HESLIN ROTHENBERG FARLEY & MESITI PC, 5 COLUMBIA CIRCLE, ALBANY, NY, 12203, US

(73) Assignee: ASPIDE MEDICAL, La Talaudiere (FR)

(21) Appl. No.: 11/751,073

(22) Filed: May 21, 2007

(30) Foreign Application Priority Data
May 24, 2006 (FR) 0651906

(51) Int. Cl. A61F 2/02

(52) U.S. Cl. 600/37; 623/11.11

(57) ABSTRACT

A prosthesis has a final shell-shaped configuration with a deformable bottom portion that can be shaped on the right side or the left side of the prosthesis thanks to its reversibility. The prosthesis has an initially flat base having at least one notch capable of receiving by overlap at least one component allowing, together with the base, the shaping of the shell-shaped prosthesis into its final configuration.

The invention relates to the technical field of anatomic prostheses for the treatment of hernias of the abovementioned type.

It is widely known how to manufacture and market anatomic prostheses for hernias which are placed by the surgeon using trocars. These prostheses must be applied perfectly to the hernia region by having a shape compatible with the implantation environment. The prostheses must be made from biocompatible, resorbable or non-resorbable materials. They are made from woven, knitted, or non-woven, non-knitted fabric, made from polypropylene, polyester or other yarns. Some prostheses are designed with assembled walls having specific shapes and profiles so that, after assembly, they provide a sort of shaping for adaptation to the implant environment. This is, for example, the case of prostheses described in patent U.S. Pat. No. 6,066,777.

Some prostheses have undergone heat treatment imparting a shape memory, described for example in patents U.S. Pat. No. 6,723,133 and FR 2,735,015.

Other hernia prostheses have thus been proposed in patents U.S. Pat. No. 6,368,541 and U.S. Pat. No. 5,954,767 with particular convex profiles.

All these hernia prostheses available on the market nevertheless have all or part of the following drawbacks.

Prostheses preshaped by heat treatment imparting a shape memory to the material have the drawback of stiffening, with the consequence of the risks of postoperative pain. Furthermore, the thermoforming operations are costly, and require specific tools and temperature control means. The shaping installation thus remains complex. All the known prostheses are configured in two implant modes, right and left, for “right” or “left” hernias of the patient, forcing the surgeon to select his prosthesis, and the staff to differentiate and manage the inventories. Moreover, these prostheses are manufactured from one and the same material and more seldom from a plurality of similar materials providing the surgeon with the same visual appearance of the prostheses during the operation. These prostheses do not offer any anatomic guide marks apart from the addition of coloured signs or bands for example, to facilitate the implant by the surgeon.

The applicant's approach has therefore been to investigate, based on the prior art corresponding to the market, the possibility of designing a novel prosthesis which overcomes these drawbacks.

The primary object was to design a single prosthesis which can be shaped and implanted both for “right” and “left” hernias of the patient, with the aim of having to manage, handle and store a single type of prosthesis.

A further object of the invention was to make a prosthesis not demanding large scale investment for its manufacture, not requiring a thermoforming phase, and which, in consequence, does not cause postoperative pain.

A further object of the invention was to improve the guidance function to aid the surgeon implanting the prosthesis.

A further object of the invention was to make a prosthesis that could be easily rolled for insertion in the implant trocar, and then adopt its final configuration, right or left, in the inguinal part to be treated.

These objects and others will clearly appear from the rest of the description.

According to a first feature, the anatomic prosthesis for treating inguinal, crural and similar hernias, of the type having a finished convex shape, is characterized in that it has a final shell-shaped configuration with a deformable bottom portion that can be shaped on the right side or the left side of the prosthesis thanks to its reversibility, and in that the said prosthesis has an initially flat base having at least one notch capable of receiving by overlap at least one component allowing, together with the base, the shaping of the shell-shaped prosthesis into its final shape.

The object of the invention is illustrated in a non-limiting manner in the figures of the drawings where:

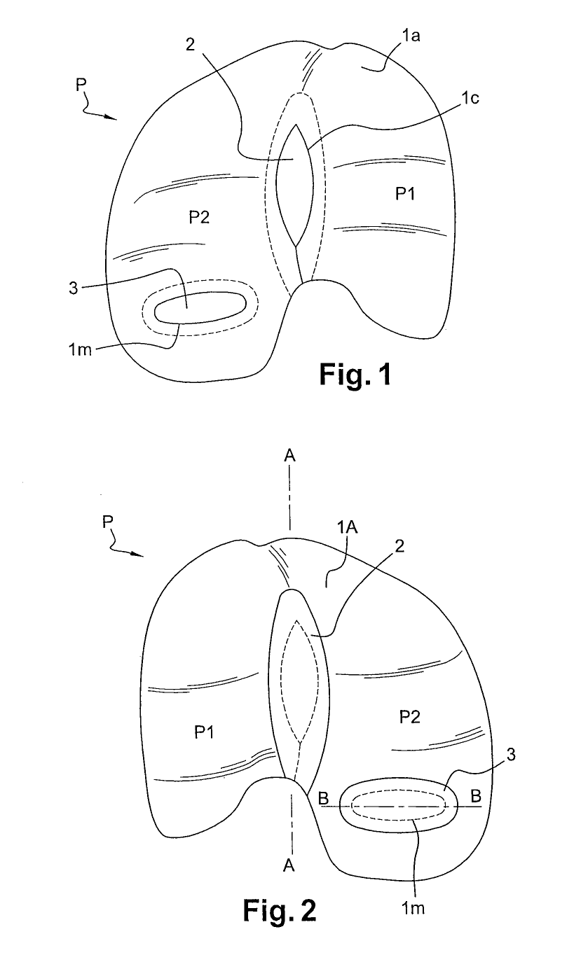

FIG. 1 is a plan view of the anatomic hernia prosthesis of the invention in a plan view,

FIG. 2 shows a perspective view of the prosthesis of the invention in its reversibility position and in its implant application for a left hernia,

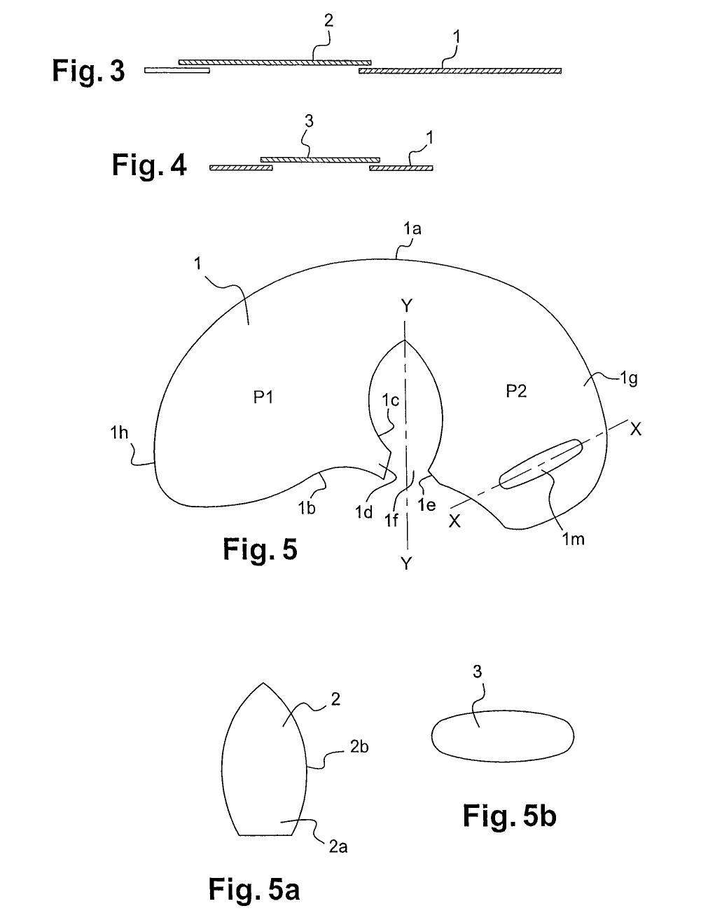

FIG. 3 shows a cross section along AA of FIG. 1 at the location of a first window,

FIG. 4 is a cross section along BB of FIG. 1 at the location of a second window,

FIG. 5 is a view of the body of the prosthesis arranged flat before final shaping of the prosthesis,

FIGS. 5a and 5b are flat views of the windows prepared, which can be positioned around the opening locations for enabling the surgeon to visualize the implant zone of the prostheses of the invention.

To make the object of the invention more concrete, it is now described in a non-limiting manner with reference to the figures.

The anatomic hernia prosthesis of the invention is referenced as a whole by (P) and has the characteristic function of being reversible and being implanted equally on the left or right side. The prosthesis (P) has a final shell-shaped configuration with a convex bottom part (1a) which may alternatively, and as required, be shaped on the right side or the left side. For this purpose, the prosthesis comprises a flat base (1) shown in FIG. 5 having a substantially crescent shape with a regularly curved outer edge (1a) and an inner edge (1b) thereby having a curved appearance but not identical to the edge (1a). An inner notch (1c) is arranged from the inner edge (1b) by defining as end portions (1d) (1e) beak shapes whereof the function will appear below. In the initial state of the prosthesis, a channel (1f) for access to the notch (1c) is therefore defined by the opposite end portions (1d) (1e). Furthermore, the base thereby defines, on either side of the said notch (1c) which is prolonged on only part of the base width, two planes (P1-P2) having a substantially different configuration, the plane P1 being more elongated than the plane (P2), with edges (1g-1h) for curved attachment to the edges (1a) and (1b).

In the initial state of the base (1) before shaping, the notch (1c) has an asymmetrical shape in the initial state that is oblong and symmetrical after final shaping of the prosthesis. Furthermore, the base (1) has, at the location of the plane (P2), a second oblong-shaped closed opening (1m) arranged in an angular plane of 70 to 100° along an XX axis with regard to the longitudinal axis YY of the notch (1c).

The base of the prosthesis thereby formed is made flat. It is capable of receiving two components (2) and (3) constituting windows having larger dimensions than the notch (1c) and the opening (1m) for superimposition thereon. These components are configured by pieces of the same material or different materials to the one constituting the base. The addition of these two components does not alter the mechanical and physical properties of the prosthesis. The component (2) is designed to be superimposed on and to surround the notch (1c) and also to be attached to the opposite sides of the end portions (1d) (1e) having a function and effect of shaping the prosthesis into its final shape by joining the portions (1d-1e). The component (3) has an oblong shape substantially larger than the opening (1m) and is attached to its perimeter.

The component (2) thereby has a heel (2a) having a shape that can overlap the two end portions (1d) (1e) for their firm attachment and joining. The heel part (2a) is prolonged by an oblong shape (2b) corresponding to the shape of the notch (1c) in order to surround it. Thereby, around the said notch (1c) and the opening (1m), the perimeter of the two components (2-3) serves to constitute windows enabling the surgeon to view the implant environment of the hernia prosthesis of the invention. The number of windows may vary.

The hernia prosthesis is shaped during the attachment of the component (2). The end portions (1d) (1e) are brought together and placed end to end, thereby creating, after attachment, a deformation of the base planes (P1) (P2), and the component (2) is attached by an ultrasonic weld for example, suture or other. The same technique is used to position the component (3) around the opening (1m) but without deforming the inner plane (P2) produced by the implant of the said component.

The hernia prosthesis of the invention thereby has a shell shape which could be imaged in the form of a shell. Thus the prosthesis, with its various assembled components (1, 2, 3) has a volumetric form that adapts to the patient's anatomy and more particularly to the abdominal cavity. The volumetric geometry of the prosthesis of the invention allows a more reliable and more anatomic positioning of the prosthesis and can help to avoid its fixing. The volumetric shell-shape confers the reversibility of use of the prosthesis, particularly by a simple manual action of placing the convex part with regard to the general plane of the prosthesis and positioning it on either side of the said plane with the convex part on one side or the other.

The prosthesis of the invention can be used by coelioscopy or laparotomy. The components (2 and 3) are joined to the base (1) preferably by ultrasound but could be attached by any other means. The materials used are, for example, polypropylene, polyester, polyurethane, silicone or PTFE. The materials are resorbable or not. The materials may also be impregnated or not with polymers, resorbable or not, in order to provide certain properties to the implant, such as tissue integration. The materials may also be made from polymer of natural origin or not, animal or not. The components (2 and 3) and the base (1) have a knitted, woven, or non-woven, non-knitted, extruded or other structure. The components (2 and 3) may have the same manufactured structure as the base or other. Preferably, the components (2 and 3) have a structure which remains aerated to permit easy and clear viewing of the prosthesis implant zone and thereby facilitating its unfolding for placement in the part of the abdomen, with the guidance of the Cooper ligament, the spermatic cord for example. Attachment may be by clipping, adhesive, or other means. The number of windows receiving the components such as components 3 may vary with a variable orientation, as required.

Thus the prosthesis has the advantage of being available in a single configuration, readily reversible right-left, contrary to the prostheses on the market. This reversibility is permitted by the manufacturing and attachment conditions of the prosthesis, which do not require the thermoforming technique.

1. Anatomic prosthesis for treating inguinal, crural and similar hernias said prosthesis being reversible and having a final shell-shaped configuration with a deformable bottom portion that can be shaped on a right side of a left side of the prosthesis thanks to its reversibility, and wherein said prosthesis has an initially flat base having at least one notch for receiving by overlap at least one component facilitating, together with the base, shaping of the shell-shaped prosthesis into its final configuration.

2. Prosthesis according to claim 1, wherein the flat base has a substantially crescent shape with beak-shaped end portions separated from one another before shaping of the prosthesis, and said end portions, when brought together, by joining with the at least one component on the base, provide anatomic shaping of the prosthesis in its final configuration.

3. Prosthesis according to claim 2, wherein the flat base has a substantially crescent shape with a regularly curved outer edge and an inner edge having a curved appearance, an inner notch being arranged and formed from the inner edge by beak-shaped end portions, and in an initial state of the prosthesis, a channel for access to the notch is defined by the opposite end portions, and wherein the base is prolonged on only part of a width of the base, and the notch divides the base into two planes having a substantially different configuration, said planes being defined with edges for curved attachment to the outer edge and inner edge of the base.

4. Prosthesis according to claim 3, wherein the notch has an oblong shape, and the base is adapted around the notch and also on sides opposite the end portions for reception and attachment of the at least one component, the at least one component having a heel to overlap the end portions and ensure firm joining and attachment of said at least one component to the base, and joining of the end portions creates a deformation of the planes of the base resulting in the final shell-shaped configuration of the prosthesis.

5. Prosthesis according to claim 2, wherein the base has, within one of the planes, a second closed oblong-shaped opening arranged in a plane perpendicular to a longitudinal axis of the notch, and a further component constituting a window is adjusted around said opening to be superimposed and attached thereto.

6. Prosthesis according to claim 5, wherein the notch and the opening each comprise a window for visualizing environment of implantation of said prosthesis.

7. Prosthesis according to claim 5, wherein the at least one component has a heel shape for overlapping the two end portions and ensuring a firm joining and attachment to the base and facilitates the redeployment of the prosthesis.

8. Prosthesis according to claim 5, wherein the at least one component and the further component are attached by an ultrasonic weld or equivalent means.

9. Prosthesis according to claims 5, wherein the base and the at least one component and the further component are made from same material.

10. Prosthesis according to claim 5, wherein the base and the at least one component and the further component are made from different materials.

11. Prosthesis according to claim 1, wherein the base is made from a material having a woven, non-woven, knitted, non-knitted, or extruded structure.

12. Prosthesis according to claim 10, wherein the at least one component and the further component are made from materials which may be woven or non-woven, knitted or non-knitted or extruded.

13. Prosthesis according to claim 5, further comprising one or more openings or windows having a variable orientation.
